(19) United States
(12) Patent Application Publication
(10) Pub. No.: US 2014/0050809 A1
(43) Pub. Date: Feb. 20, 2014

(54) Non-Toxic Pest Repellent for Use on Humans/Animals

(71) Applicant: Jennifer Smith Angone, Berwyn, IL (US)

(72) Inventor: Jennifer Smith Angone, Berwyn, IL (US)

(73) Assignee: Jennifer Smith Angone, Berwyn, IL (US)

(21) Appl. No.: 13/966,533

(22) Filed: Aug. 14, 2013

Related U.S. Application Data
(60) Provisional application No. 61/683,870, filed on Aug. 16, 2012.

(51) Int. Cl. A61K 36/899; A61K 36/13; A61K 36/53; A61K 36/185

(52) U.S. Cl. CPC A61K 36/899; A61K 36/185; A61K 36/13; A61K 36/53
USPC 424/770

(57) ABSTRACT

This invention relates generally to a non-toxic insect repellent comprising natural ingredients for application on humans and or animals. More particularly, the present invention is directed toward a non-aerosol insect repellent for application to the skin or clothing of humans and animals to repel pests found in a variety of environments, such as mosquitoes, black flies, ticks, and biting gnats. The present invention comprises the active ingredients of citronella oil, lemongrass oil, geranium oil, cedarwood oil, rosemary oil, and sesame oil that work in combination to repel pests from humans and animals over an extended period of time. The present invention also comprises an emulsifier and a preservative to provide extended shelf-life. Additionally, the present invention may optionally contain catnip oil for enhanced potency to repel biting infectious insects, particularly when used in more wilderness areas.

### FIELD OF THE INVENTION

This invention relates generally to a non-toxic insect repellent comprising natural ingredients for application on humans and or animals. More particularly, the present invention is directed toward a non-aerosol insect repellent for application to the skin or clothing of humans and animals to repel pests found in a variety of environments, such as mosquitoes, ticks, black flies, & biting gnats. The present invention comprises the active ingredients of citronella oil, lemongrass oil, geranium oil, cedarwood oil, rosemary oil, and sesame oil that work in combination to repel pests from humans and animals over an extended period of time. Additionally, the present invention may optionally contain catnip oil for enhanced potency to repel biting insects, particularly when used in heavy wilderness areas.

### BACKGROUND OF THE INVENTION

Pests have long been a nuisance to humans and animals, particularly when spending time in outdoor environments. Although humans seek an effective solution for repelling pests from themselves and their pets, many individuals are concerned about introducing repellents containing high levels of toxic chemicals on or near their skin or clothing or their pets' skin. Over time it has become apparent that widespread use of synthetic chemical pesticides may be harmful to humans and animals, including the chemical commonly known as DEET (N,N-diethyl-m-toluamide) that is found in many repellents on the market. As a result, natural and organic pesticides are of great interest because of their safety to humans and pets, including compositions that do not contain DEET.

Additionally, it is desirable that a pest repellent be effective in a variety of environments, such as backyards and/or wilderness areas, in order to provide an effective, all-around solution for users to repel a variety of pests. A pest repellent should further last for extended periods of time to reduce the need for repeated applications. As many users are sensitive to the chemical smells and the effects of those chemicals, particularly when applied to the skin or clothing, it is also desirable for a pest repellent to have a pleasant, unobtrusive fresh scent.

Additionally, for environmental purposes, it is desirable that the repellent be applied via an environmentally friendly means. Aerosol sprays may have harmful consequences to the environment because chlorofluorocarbons (CFCs) are found to dissolve the protective Ozone Layer. Without the Ozone layer, certain damaging rays from our sun make it to earth and causes global warming and skin cancer (etc . . . ). Additionally, the moisture may cause aerosol cans to rust, resulting in the presence of potentially harmful contents. Pressurized cans also raise concerns regarding their safe disposal. Thus, it is desirable for a pest repellent to be stored in an easily recyclable container and dispersed using a non-aerosol pump and/or wipe delivery system.

Essential oils have been found to be effective in the deterrence of pests because of certain properties. For example, oils such as lemongrass and citronella contain monoterpene aldehydes, which have been found to be effective in repelling insects. Additionally, cedarwood oil, which contains, among other components, cedrene, b-cedrene, thujopsene, cederol, and widdrol, has been found to be an effective pest repellent in locations where cedarwood is present. Combinations of essential oils, such as geranium oil and lemongrass oil, are known to further enhance the effectiveness of essential oils in repelling insects. These and other features of essential oils applicable to deterring pests will be appreciated by one of ordinary skill in the art.

Further, it will be known to one of ordinary skill in the art that carrier oils are also very effective in repelling pests. For example, sesame oil is effective as a pest repellent because, among other properties, sesame oil has a high viscosity which prevents the insect from moving upon application of the oil. Sesame oil also has the favorable property of being safe for use in proximity of both humans and animals.

The current invention, not previously seen in the prior art, is a non-toxic, DEET-free pest repellent for use by humans or animals that repels pests found in various environments, such as biting flies, over an extended period of time containing the active ingredients of citronella oil, lemongrass oil, geranium oil, cedarwood oil, rosemary oil, and optionally catnip oil, in admixture with a carrier and a dispersant.

In reviewing the prior art, various pest repellents containing essential oils are disclosed in efforts to repel insects on humans and animals using natural or organic means. U.S. Pat. No. 1,995,247 issued to Haring et al. discloses that the products of aromatic lactones, such as cumarins, are particulary effective as insect repellents. The cumarin compounds may be combined with semi-solid materials such as creams, jellies, and soaps. Haring '247 does not disclose a spray insect repellent comprising a combination of essential oils including sesame oil, citronella oil, lemongrass oil, geranium oil, cedarwood oil, rosemary oil and optionally catnip oil.

Similarly, U.S. Pat. No. 4,178,384 issued to Ensing et al. discloses a long lasting insect repellent comprising a low concentration of pyrethroid insecticide as the active component in admixture with a carrier to repel cockroaches, flies, beetles, and moths from, for example, areas where food is present. Unlike the present invention, Ensing '384 does not disclose an insect repellent spray directed toward application on humans and animals.

Another disclosure, U.S. Pat. No. 5,565,208 to Vlasbom et al. which discloses a topical insect repellent for humans and animals comprising d-limonene, di-N-proply isocinchomeronate, N-octyl bicycloheptene dicarboximide, and other chemicals where the composition has been found to last for up to two full days. Vlasbom '208 does not disclose an insect repellent directed for both humans and animals comprising a combination of essential oils, including sesame oil, citronella oil, lemongrass oil, geranium oil, cedarwood oil, rosemary oil, and optionally catnip oil.

Another disclosure, U.S. Pat. No. 5,575,988 to Knowles, Jr. et al. discloses a combination sunscreen and insect repellent in a lotion or cream form, free of organic chemical sunscreen, but containing DEET. Knowles, Jr. et al. '988 is thus distinguishable from the present invention which comprises a DEET-free liquid spray pest repellent.

U.S. Pat. No. 5,589,181 issued to Bencsits discloses a topical insect repellent comprising of at least one fatty acid alkyl ester, at least one natural or synthetic fatty alcohol serving as the active substance, and at least one natural vegetal or animal fatty oil as the carrier. Bencsits '181 discloses that the fatty acid alkyl ester is preferably an ester of fatty acids selected from rape-seed oil, sunflower oil, peanut oil, peanut butter, soy oil, safflower seed oil, cuphea oil, coconut oil, palm kernel oil, palm oil, and fish oil. Bencsits '181 does not disclose an insect repellent comprising sesame oil, citronella oil, lemongrass oil, geranium oil, cedarwood oil, rosemary oil, and optionally catnip oil.

U.S. Pat. No. 5,621,013 issued to Beldock et al. discloses insect repellent lotions and sprays comprising citronella, geraniol, crystalline 3,8 P-menthanediol (chinese crystal) as the plant derivative responsible for effective insect repelling properties, and terpineol or rhodinol. Beldoct et al. '013 does not disclose an insect repellent comprising sesame oil, citronella oil, lemongrass oil, geranium oil, cedarwood oil, rosemary oil, and optionally, catnip oil.

U.S. Pat. No. 5,776,478 issued to Jain discloses a topical insect repellent comprising a mixture of lemongrass oil and cedarwood oil, a solvent preferably in the form of an alcohol, and preferably an antioxidant to serve as a stabilizer, such as a mixture of ascorbic acid and citric acid (e.g., lemon juice and lime juice). The composition in Jain '478 is described to perform three functions, namely, repelling insects, protecting the skin from UV rays and providing post-bite relief. Jain '478 does not disclose an insect repellent comprising a combination of several essential oils including sesame oil, citronella oil, lemongrass oil, geranium oil, cedarwood oil, rosemary oil, and optionally, catnip oil.

U.S. Pat. No. 5,885,600 issued to Blum et al. discloses a composition with insect repellent properties comprising a combination of neem, citronella, and cedarwood oils. An antioxidant such as buylated hydroxytoluene is added to the essential oils to preserve efficacy. Blum et al. '600 discloses that the resulting composition may be applied as lotions, creams, or sprays to humans and vegetation or formed into polymeric sheets which then may be formed into articles such as animal collars or garbage bags or woven into clothing. Blum et al. '600 does not disclose a non-toxic insect repellent developed as a liquid spray for humans and pets comprising a combination of essential oils, including sesame oil, citronella oil, lemongrass oil, geranium oil, cedarwood oil, rosemary oil, and optionally, catnip oil.

U.S. Pat. No. 6,306,415 issued to Reifenrath et al. discloses a method for repelling flies from humans by applying topical insect repellent made from fatty acids or other organic molecular species typically found on human skin that is approximately as effective as DEET. The carrier disclosed in Reifanrath may comprise silicone. The composition disclosed in Reifenrath '415 does not disclose a combination of sesame oil, citronella oil, lemongrass oil, geranium oil, cedarwood oil, rosemary oil, and optionally, catnip oil.

U.S. Pat. No. 6,808,717 issued to Bale et al. discloses an aerosol coolant spray dispersed from a pressurized aerosol spray canister for immobilizing and killing ticks from an area of the human skin. The aerosol coolant spray disclosed by Bale '717 comprises a coolant material, an essential oil, and a diluent material. Bale '717 discloses that the essential oil may be selected from the group consisting of cedar oil, citronella oil, wintergreen oil, geranium oil, lemongrass oil, rosemary oil, and the like. Bale '717 further discloses the diluent material may include polysorbate 20/60/80, glyceryl cocoate, alginate, gum arabig and the like. Bale '717 does not disclose, however, a composition for repelling mosquitoes, black flies, ticks, biting gnats comprising a combination of sesame oil, citronella oil, lemongrass oil, geranium oil, cedarwood oil, rosemary oil, and optionally, catnip oil.

U.S. Pat. No. 6,811,789 issued to Sakurai et al. discloses a fragrant composition exhibiting mosquito-repellent properties almost equal to the effect of DEET, does not irritate human skin, is safe for the environment and is comprised of a mixture of isomethone, linalool, geraniol, citral, and citronellol. Sakurai '789 further discloses the composition may further comprise a fatty acid having 8 to 10 carbon atoms such as octanoic acid, nonannic acid, and the like. Sakurai '789 is directed toward mosquito repellent whereas the present invention repels mosquitoes, black flies, ticks, and biting gnats. Further the composition in Sakurai '789 does not comprise a combination of sesame oil, citronella oil, lemongrass oil, geranium oil, cedarwood oil, rosemary oil, and optionally, catnip oil.

Similarly, U.S. Pat. No. 6,936,269 issued to Robinson discloses an insect repellent substrate for use in repelling and killing lice comprising of wax, such as paraffin, and a natural insect repellent such as pyrethrum oil, and may include other oils such as citronella oil, rosemary oil, and neem oil, comprise a fabric base made of felt comprised of this repellent composition to form a headband or cap. Robinson '269 does not disclose a liquid spray insect repellent for repelling flies, mosquitoes, and other insects from humans and pets comprising a combination of essential oils, including sesame oil, citronella oil, lemongrass oil, geranium oil, cedarwood oil, rosemary oil, and optionally, catnip oil.

U.S. Pat. No. 7,067,678 issued to Scialdone discloses the preparation of a derivative of dihydronepetalactone prepared by the deprotonation of nepetalactone (catnip oil) and treatment with Grignard reagent. Scialdone discloses that the resulting odorous compound may be used in insect repellents. Unlike the present invention, the composition disclosed in Scialdone is derived from catnip oil. Scialdone does not disclose an insect repellent comprising a combination of essential oils, including sesame oil, citronella oil, lemongrass oil, geranium oil, cedarwood oil, rosemary oil, and optionally, catnip oil.

U.S. Pat. No. 7,201,926 issued to Fried et al. discloses an insect repellent for humans comprising of citronella oil, D-limonene, at least two synergists, and at least three essential oils. Fried et al. '926 discloses that it is the combination of citronella oil with D-limonene and essential oils that gives the composition its unique insect repelling properties. Further, Fried '926 further discloses the aldehydes and preferred essential oils are geranium oil, rosemary oil, and peppermint oil, although other essential oils with insect properties may be used, including lavender oil and eucalyptus oil as well as terpene. Fried et al '926 does not disclose however a repellent comprising sesame oil, citronella oil, lemongrass oil, geranium oil, cedarwood oil, rosemary oil directed for use on humans and animals.

U.S. Pat. No. 7,344,728 issued to Perry which discloses an insect repellent composition for humans, pets, and livestock comprising natural or synthetic ingredients, sunscreen, volatile silicones, and a film forming polymer to increase water resistance. Perry '728 discloses that the composition may include a blend of citronella oil, orange oil, camphor oil, cedarwood oil, and the like or comprise synthetics such as linalyl acetate, cineole, or phthalic acid. The composition disclosed in Perry '728 does not comprise a combination of primarily natural oils including sesame oil, citronella oil, lemongrass oil, geranium oil, cedarwood oil, and rosemary oil. Unlike the composition disclosed in Perry '728 the natural oils in the present invention are not intended to be replaced with synthetic ingredients.

Similarly, U.S. Pat. No. 7,575,765 issued to Hughes discloses an all-natural topical insect repellent with a carrier base of fractionated coconut oil and the following essential oils in the following percentages by weight are included: cedarwood in the range of 1.36 to 1.74%, citronella oil in the range of 3.55 to 3.99% and lemongrass oil in the range of 9.02 to 9.57%. Unlike the present invention the topical pest repellent does not contain sesame oil as a carrier, nor does it contain geranium oil or rosemary oil or optionally, catnip oil.

U.S. Pat. No. 7,902,256 issued to Ping discloses a pediculicidal and ovicidal composition that may be applied topically to humans, animals or infested areas. The composition in Ping '256 contains a purified terpene agent, such as citronellal or citronellyl, a short chain alcohol such as ethanol, and a surfactant. The disclosure in Ping '256 is particularly directed toward the removal of parasites such as lice and their ova and does not comprise a combination of sesame oil, citronella oil, lemongrass oil, geranium oil, cedarwood oil, rosemary oil, potassium sorbate and polyglyceryl oleate directed at repelling a variety of insects, including mosquitoes, black flies, ticks, and biting gnats.

U.S. Pat. No. 7,947,311 issued to Porter et al. discloses a non-toxic insect repellent comprising essential oils and at least one emulsifying agent containing all-natural ingredients. The composition disclosed in Porter '311 includes a pH conditioning agent, cinnamon leaf oil, garlic oil, lemongrass oil, peppermint oil, citronella oil, lecithin, soybean oil, and apple cider vinegar. The composition disclosed in Porter '311 further discloses that soybean oil may serve as both an all-natural emulsifying agent and buoyancy agent that also can suffocate insect larvae. Porter '311 however does not disclose a pest repellent spray containing sesame oil, citronella oil, lemongrass oil, geranium oil, cedarwood oil, rosemary oil, and optionally, catnip oil.

U.S. Pat. No. 7,988,985 issued to Bessette discloses a topical insect repellent containing a carrier made of isopropyl myristate and isopropyl alcohol, and an active ingredient comprising rosemary oil, cinnamon leaf oil, lemongrass oil, wintergreen oil and geranoil. The composition disclosed in Bessette '985 further discloses the use of other plant essential oils in the composition including rosemary, thyme, peppermint oil, and sesame oil. But Bessette '985 does not disclose a pest repellent spray containing sesame oil, citronella oil, lemongrass oil, geranium oil, cedarwood oil, rosemary oil, and optionally, catnip oil.

Thus, no where in the prior art is seen a non-toxic pest repellent effective for use on humans and animals that effectively repels against mosquitoes, black flies, ticks, and biting gnats over a 4-5 hour period of time containing the active ingredients of sesame oil, citronella oil, lemongrass oil, geranium oil, cedarwood oil, rosemary oil, in admixture with the sesame carrier oil, polyglyceryl oleate as the emulsifier, and potassium sorbate as the all-natural preservative.

### SUMMARY OF THE INVENTION

The present invention consists a non-toxic pest repellent that effectively repels pests found in a variety of outdoor environments, such as mosquitoes, black flies, ticks, and biting gnats, upon external application on humans and animals. The present invention comprises the active ingredients of citronella oil, lemongrass oil, geranium oil, cedarwood oil, rosemary oil, and sesame oil that work in combination to repel pests from humans and animals over an extended period of time. Additionally, inactive ingredients potassium sorbate serves as the all-natural preservative and polyglyceryl oleate serves as the vegetable-based emulsifying agent. Further, the present invention may optionally contain catnip oil for enhanced potency to repel biting insects and ticks in more wilderness areas. It will be known to one of ordinary skill in the art that the plant essential oils and their derivatives exhibit insecticidal repellent properties. Whereas essential oils such as lemongrass oil and citronella oil may serve as effective repellents on their own, it is the unique combination of all the oils in the present invention that gives the present invention that gives the present invention its ability to effectively repel a variety of pests from humans and animals over an extended period of time while emitting a unique fresh scent that is pleasant to the user and passerby. The present invention is preferably applied via an environmentally-friendly non-aerosol pump spray and alternatively, via a wipe delivery system.

The present invention comprises a carrier oil such as sesame oil to effectively and safely deliver the active essential oil ingredients and their natural insect repelling properties to the skin and clothing on humans and fur/hair on animals. The present invention further preferably comprises an emulsifier to evenly suspend the mixture of oils which in this composition is polyglyceryl oleate. Further, the present invention also includes potassium sorbate as the natural preservative and a dispersant to facilitate even delivery of the oils when applied to target areas requiring protection. In a preferred embodiment, the present invention comprises ingredients known to one of ordinary skill in the art that serve to preserve the present invention.

### OBJECTS OF THE INVENTION

Thus, it is one primary object of the present invention to provide a non-toxic pest repellent that repels pests comprising a carrier oil.

It is another primary object of the present invention to provide a non-toxic pest repellent that may be applied as a non-aerosol spray formulation for external use on humans and animals comprising a dispersant to facilitate distribution of the present invention.

It is another primary object of the present invention to provide a non-toxic pest repellent that repels pests from humans and animals in a variety of outdoor environments such as backyards and hiking trails, boating and fishing, comprising a carrier oil, a dispersant, a preservative, and emulsifier and a combination of essential oils comprising citronella oil, lemongrass oil, cedarwood oil, geranium oil and rosemary oil in admixture with carrier, a dispersant, preservative and emulsifier.

It is another primary object of the present invention to provide a non-toxic pest repellent that repels pests over a period of time comprising a carrier oil, a dispersant, and a combination of essential oils comprising citronella oil, lemongrass oil, geranium oil, cedarwood oil, rosemary oil in admixture with the carrier, a dispersant, preservative, and emulsifier.

It is another primary object of the present invention to provide a non-toxic pest repellent with enhanced repellent properties for additional protection from biting and infectious insects comprising a carrier oil, a dispersant, preservative, emulsifier, and a combination of essential oils comprising citronella oil, lemongrass oil, cedarwood oil, geranium oil, and catnip oil.

It is yet another primary object of the present invention to provide a non-toxic pest repellent with a pleasant, unobtrusive scent comprising a combination of essential oils including citronella oil, lemongrass oil, cedarwood oil, geranium oil, rosemary oil, and optionally, catnip oil.

It is yet another primary object of the present invention to provide a non-toxic pest repellent that is free of the chemical commonly known as DEET comprising a carrier oil, a dispersant/preservative, an emulsifier, and a combination of essential oils comprising citronella oil, lemongrass oil, cedarwood oil, geranium oil, rosemary oil, and optionally catnip oil.

It is yet another primary object of the present invention to provide a non-toxic pest repellent that provides extended pest repellent properties upon application comprising a carrier oil, a dispersant, an emulsifier, and a combination of essential oils comprising citronella oil, lemongrass oil, cedarwood oil, geranium oil, rosemary oil, and optionally catnip oil in admixture with the carrier (sesame oil), preservative (potassium sorbate) and emulsifier (polyglyceryl oleate).

It is yet another primary object of the present invention to provide a non-toxic pest repellent as a spray formulation and alternatively, as a wipe, wherein the pest repellent further comprises an emulsifier, polyglyceryl oleate, to promote a stable combination of oils throughout the dispersant. concentrated combination of oils to be evenly suspended throughout the dispersant. In a preferred embodiment, the emulsifying agent is classified as GRAS (Generally Recognized As Safe) by the FDA.

It is yet another primary object of the present invention to provide a non-toxic pest repellent having a stable shelf life further comprising the preservative potassium sorbate.

These and other objects and advantages of the present invention can be readily derived from the following detailed description of the preferred embodiments and should be considered as within the overall scope of the invention.

### DETAILED DESCRIPTION OF THE PREFERRED EMBODIMENTS

The present invention relates to an non-toxic pest repellent that repels pests such as mosquitoes, black flies, ticks and biting gnats from humans and animals for extended periods of time. The present invention is effective in a variety of outdoor environments including backyards as well as more wilderness areas such as hiking trails, boating and/or fishing on lakes, oceans, docks & marinas. The repellent composition comprises the active ingredients of lemongrass oil, citronella oil, cedarwood oil, geranium oil rosemary oil, and optionally catnip oil, in admixture with a carrier oil and a dispersant. The present invention preferably contains the preservative and an emulsifier and is applied to skin or clothing via a non-aerosol pump spray and/or wipe system of delivery.

In a preferred embodiment, the present invention comprises ingredients in the approximate amounts be percent of weight as provided in Table 1.

TABLE 1
Ingredients in Approximate Percent by Weight

| Ingredient | Approximate Percent by Weight |
|---|---|
| Citronella Oil | 7% |
| Lemongrass Oil | 5% |
| Geranium Oil | 4% |
| Cedarwood Oil | 3% |
| Rosemary Oil | 2% |
| Carrier Oil (sesame oil) | 8% |
| Dispersant (water) | 69% |
| Preservative (potassium sorbate) | .05% |
| Emulsifier (polyglyceryl oleate) | 1.5% |

In a preferred embodiment, the carrier oil is sesame oil, the dispersant is water. the preservative is potassium sorbate, and the emulsifier is polyglyceryl oleate.

The present invention may optionally include catnip oil to provide enhanced protection against biting infectious insects that users may encounter on longer more dense outdoor adventures and environments. In a preferred embodiment, the invention containing the optional addition of catnip oil comprises ingredients in the approximate amounts by percent of weight as provided in Table 2.

TABLE 2
Ingredients in Approximate Percent by Weight

| Ingredient | Approximate Percent by Weight |
|---|---|
| Citronella Oil | 7% |
| Catnip Oil | 5%-7% |
| Lemongrass Oil | 3%-4% |
| Geranium Oil | 2%-3% |
| Cedarwood Oil | 1%-2% |
| Rosemary Oil | 2% |
| Carrier Oil | 8% |
| Dispersant | 69% |
| Preservative | .05% |
| Emulsifier | 1.5% |

Although in the foregoing detailed description the present invention has been described by reference to various specific embodiments, it is to be understood that modifications and alterations in the structure and arrangement of those embodiments other than those specifically set forth herein may be achieved by those skilled in the art and that such modifications and alterations are to be considered as within the overall scope of this invention.

As used herein, the term “repel” refers to the minimization of the presence of a targeted pest(s) within a targeted area. “Repel” may also refer to the deterrence of the targeted pest(s) from entering or remaining in a targeted area.

As used herein, the term “outdoor environment” includes, but is not limited to backyards, forests, hiking trails, boat docks, boats, seafaring vessels, sporting events, public concerts, picnics, playing sports, fishing, hunting, bicycling, gardening, golfing and like outdoor interests.

As used herein, the term “essential oil” refers to oils obtained from the root, bark, stem, leaves, or aromatic portions of plants.

As used herein, the term “carrier oil” refers to any oil obtained from the fatty portion of plants, such as seeds or nuts, with which the essential oils are mixed and which serves to dilute the concentration of the essential oils. The term “carrier oil” may also be commonly referred to by those of ordinary skill in the art as base oils or fixed oils.

What is claimed is:

1. A non-toxic pest repellent for external use by humans and animals to repel pests, including mosquitoes, black flies, ticks, and biting gnats in a variety of environments comprising:
a carrier oil in an amount of about 8% by weight,
a dispersant,
a combination of essential oils comprising citronella oil in an amount of about 7% by weight, lemongrass oil in an amount of about 5% by weight, geranium oil in an amount of substantially 4% by weight, cedarwood oil in an amount of about 3% by weight, and rosemary oil in an amount of about 2% by weight, wherein the combination of essential oils are in admixture with the carrier oil and the dispersant.

2. The non-toxic pest repellent of claim 1, wherein the carrier oil is sesame oil.

3. The non-toxic pest repellent of claim 1, wherein the dispersant is water.

4. The non-toxic pest repellent of claim 1, further comprising a preservative.

5. The non-toxic pest repellent of claim 4, wherein the preservative is present in an amount of about 0.05% by weight.

6. The non-toxic pest repellent of claim 4, wherein the preservative is potassium sorbate.

7. The non-toxic pest repellent of claim 1, further comprising an emulsifier.

8. The non-toxic pest repellent of claim 7, wherein the emulsifier is present in an amount of about 1.5% by weight.

9. The non-toxic pest repellent of claim 7, wherein the emulsifier is polyglyceryl oleate.
